(19) United States
(12) Patent Application Publication
(10) Pub. No.: US 2002/0048776 A1
(43) Pub. Date: Apr. 25, 2002

(54) Determination of ligands for proteins

(76) Inventors: Cornelius Frommel, Zeuthen (DE); Robert Preissner, Berlin (DE); Andrean Goede, Berlin (DE)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/772,538

(22) Filed: Jan. 29, 2001

(30) Foreign Application Priority Data
Jul. 15, 1998 (DE) 19831758.1

(51) Int. Cl. G01N 33/53; G01N 33/542; G01N 21/00; C12Q 1/00

(52) U.S. Cl. 435/7.9; 422/55; 435/4

(57) ABSTRACT

This invention relates to a method for determining ligands for proteins. Said method comprises determining, by means of secondary structural elements of a given protein which form the binding site, molecular surface patches which are compared with known molecular surface patches with ligand.

### RELATED APPLICATIONS

This application is a continuation in part of U.S. application Ser. No. 09/***,*** which is the U.S. National Phase application under 35 U.S.C. §371 of International Application PCT/EP99/04951, filed Jul. 13, 1999, which claims priority of German Application DE 198 31 758.1, filed Jul. 15, 1998.

### BACKGROUND OF THE INVENTION

1. Field of the Invention

This invention relates to a process to determine ligands for proteins according to the following steps: determining the secondary structural elements of a given protein that constitute the binding site for the ligand; breaking down the molecular surface of the protein into molecular surface elements; determining surfaces similar to those surface elements that define the binding region for the ligand that is to be determined, whereby the molecular surface patches found have a complementary neighboring element; coordinate transformation of the molecular surface patches with neighboring elements that have been found, based on a starting element, and at an rms value less than 2A; assessment of the fit of the ligands in terms of local packing density.

2. Description of the Related Art

In biochemistry ligands are understood to be generally low-molecular weight, biologically active substances that exert a particular effect on a macromolecule by binding to a specific binding site on the macromolecule. The macromolecules in question here may be proteins such as enzymes, receptors, structural proteins, transcription factors, signal transduction proteins, as well as, nucleotide molecules including, DNA, RNA etc.

It is possible, for example, by binding of a ligand to a macromolecule to achieve effects such as catalytic conversion of an enzyme, activation or inactivation of an enzyme, inhibition of a protein-protein interaction or conformational changes of macromolecules.

Two strategies have been employed to date in the pharmaceutical industry to identify biologically active substances i.e. ligands.

Companies generally have large repositories of many different compounds. These substances are assayed for specific activities in biological systems e.g. cell assays using high throughput methods. One example of such an assay method uses pipetting lines with automatic evaluation. Suitable molecules are only found by chance using this method, however there is a certain degree of probability that such molecules will occur.

An alternative to this approach is a strategy using computers. Based on calculation of the fit and the forces between molecules, compounds to bind with specific protein surfaces can be modeled virtually on a computer and then synthesized. In contrast with the aforementioned assay methods, fewer substances are required to be synthesized and tested. Virtual substance libraries of molecules, which do not need to be present as physical substances, can be tested in a docking simulation on the computer to determine whether they bind with a particular protein surface. Here again only the suitable substances discovered to yield a desirable activity are synthesized and employed in biological test systems. Processes of this type have already been described in U.S. Pat. Nos. 5,495,423, 5,579,250 and 5,612,895.

In practice, combinations of the processes described above are often used.

In these processes, in-vivo or naturally occurring interactions may not be accurately assessed. Furthermore, many known processes are subject to complex interactions and conditions which may be observed only through repeated experimentation and virtual observations. This makes the procedure lengthy and causes a high degree of imprecision.

### SUMMARY OF THE INVENTION

This invention therefore seeks to solve the problem of making a process available to determine ligands for proteins rapidly and reliably.

This problem is solved in a process according to determine the ligands for proteins, comprising the following steps: determining the secondary structural elements of a given protein that constitute the binding site for the ligand; breaking down the molecular surface of the protein into molecular surface elements; determining surfaces similar to those surface elements that define the binding region for the ligand that is to be determined, whereby the molecular surface patches found have a complementary neighboring element; coordinate transformation of the molecular surface patches with neighboring elements that have been found, based on a starting element, and at an rms value less than 2A; assessment of the fit of the ligands in terms of local packing density.

The dependent claims relate to preferred embodiments of the process according to the invention.

### BRIEF DESCRIPTION OF THE DRAWINGS

FIG. 1 is a flow diagram illustrating a sequence of steps used to determine suitable ligands for protein interaction.

FIG. 2 is a block diagram which illustrates the use of a database of structural elements to determine suitable ligands.

### DETAILED DESCRIPTION OF THE PREFERRED EMBODIMENT

The process to determine ligands for proteins according to the invention comprises the following steps:

a) Determining those secondary structural elements of a particular target protein that constitute the binding site for the ligand. In particular, a surface area of the particular protein, which constitutes a binding site for the ligand to be predicted, is determined.

b) Breaking down the molecular surface of the given target protein into molecular surface elements. In particular, the secondary structural elements in a three-dimensional model of the given target protein are defined in terms of hydrogen bonds, whereby, as a function of the surface area determined in a), adjacent secondary structures, relative to the binding site, may also be surmised. Furthermore, large secondary elements that project beyond the surface area of the binding site may also be modeled and divided. The molecular surface element thus is representative of the target protein to which the ligand has been determined to bind and is built up by secondary structural elements derived from the target protein.

c) Determining known molecular surface patches (basis patches having surface areas of secondary structural elements made of groups of atoms) similar to those molecular surface elements that define the binding site for the ligand, whereby the basis patches identified have a complementary moleculae surface patch (contact patch). In particular, atoms exposed to a surrounding solvent in each of the secondary structural elements belonging to a surface area as defined in a), build up the molecular surface elements for that ligand to define search surfaces. The atoms are determined by scanning the surface with a water molecule model on a Connolly surface.

A basic set of search data pairs of surfaces are determined (basis patch/contact patch pairs: together defined as an interacting surface pair) which are in contact with each other using all or part of proteins or protein complexes with a known three-dimensional structure. The models of the proteins are subsequently broken down into secondary structural elements and parts of the secondary structural elements on the basis of the hydrogen bonds or other geometric parameters. This process is aided by a determination of the atoms of a secondary structural element, namely the contact surface, which are within a Van der Waals distance from another pairing secondary structural element or from the surrounding solvent.

One entry of the basic set of search data comprises two interacting secondary structural elements whereby the contacts are formed only by the contacting parts of their surface (basis patch and surface patch). In contrast to other approaches, the process described here includes the pairs of interacting secondary structural elements from a single protein in addition to those from protein-protein complexes whereby the basis patch is derived from one protein and the contact patch from the other protein. Thus, the number of entries for the basic set of search data is up to 6,000,000 in contrast to 8,000 for those derived from protein-protein interactions (numbers calculated on the basis of entries contained in the Protein Data Bank).

In particular, basis patches are determined to be similar to those molecular surface elements that define the binding site for the ligand, whereby the basis patches found have a complementary neighboring element (contact patch). The center and maximum extent of the molecular surface elements are superimposed on all or part of the basis patches wherein the superimposition may be optimized by maximizing the atoms superposed and minimizing the root-mean-square deviation.

d) Co-ordinate transformation is effected on the basis patches found together with the corresponding contact patches on molecular surface elements that are defined in a) and b) with an rms value of less than 2A. In particular a coordinate transformation is done to transform the surface found into the search area for given proteins.

e) Assessment of the fit of the contact patches with the molecular surface elements as defined in a) and b) in terms of local packing density. In addition, superimposition of the basis patch with the molecular surface elements is carried out with respect to the number of superimposed atoms, the number of superimposed atoms of the same atomic type and the root-mean-square deviation. A correlation may be assessed in terms of the local packing density as determined by a comparison between the surface found and the given protein.

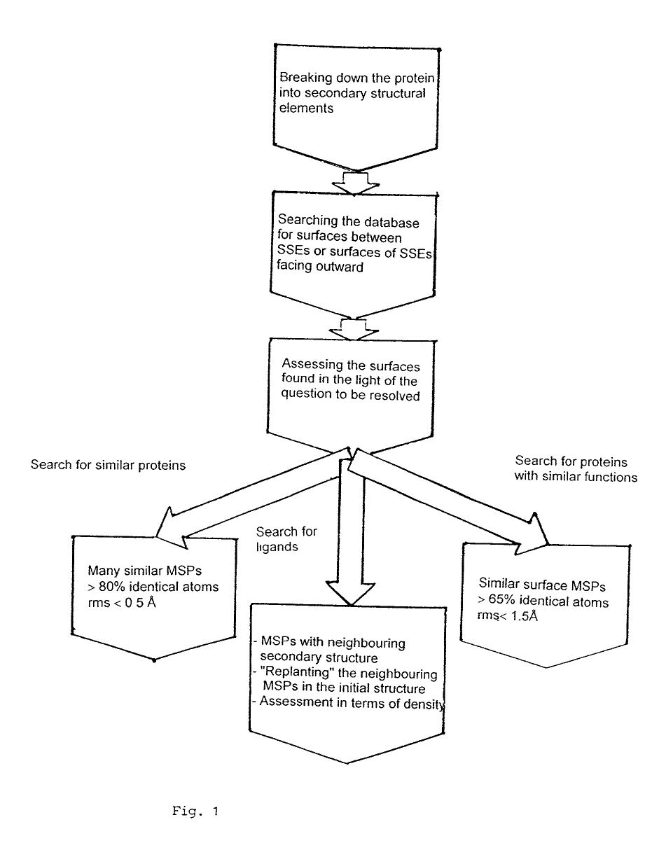

The sequence of steps in the process according to the invention is shown in the flow diagram in FIG. 1.

The process according to the invention is preferably carried out using a database, particularly after step e). It has proved to be advantageous to use the database “Dictionary of Interfaces in Proteins (DIP)”, Journal of Molecular Biology, Vol. 280, p. 535 ff., 1998. The DIP database makes available the interacting surface pairs between secondary structural elements of all proteins whose structure is known. These interfaces are made up of two groups of atoms (patches), which are part of neighboring secondary structures and together constitute the contact between these two structures (basis patch and contact patch).

In determining ligands for purposes such as drug design, the question arises of which chemical compound fits a given protein structure. According to the invention, the process begins at a step wherein, for a given target protein the secondary structural elements that constitute the binding site for the ligand are determined. Next the molecular surface for the protein is broken down into molecular surface elements.

Surfaces similar to those elements that potentially define the binding region are selected (basis patches), for example from the database described above. A further condition is required in similarity screening, namely that the basis patches found already have a complementary neighboring element. If the rms value (mean error) is less than 2A, it may be helpful to carry out a transformation, for example, a coordinate transformation, of the basis patch found together with its contact patch on the initial molecular surface element. The rms value is preferably 1.5 A. The most useful way to appraise the fit of the ligand compared with the original has proved to involve using the local packing density as defined by Goede et al., Journal of Computational Chemistry, Volume 18, No. 9, p. 1114 ff., 1997.

According to the invention, the external surfaces of a secondary structural element are to be determined. The external surfaces that establish contact are the molecular surface elements. Similar basis patches are superimposed. After the coordinate transformation, the basis patches found lie on atoms of the binding site. The best potential ligands constitute the lead compound. The last step is to compare the best potential ligands with a known starting protein plus ligand.

Thus, according to the invention a complementary binding partner is determined by determining similar elements that already have a binding partner.

After determination of the ligands, which are secondary structural elements made up of around 10 amino acids, these ligands must be optimized before they can be used as medicaments, for example, as peptides made up of natural L-amino acids fail to meet a number of requirements in this respect.

Experimental processes exist for synthetic transformation of peptides into peptidomimetics e.g. peptoides, which often have much more favorable qualities from a pharmacological perspective. The compounds generally undergo a number of optimization cycles using focused compound libraries derived from the initially identified ligand with the compounds present as substances as well as modeling approaches.

Another option that may be employed to find lead compounds involves searching databases of low-molecular compounds. In this case, the coordinates of a peptide ligand that offers a good fit or its pharmacological relevant groups (pharmacophor) are used to run a search in a suitable database using the superposition method described above (comparative process). This makes it possible to find lead compounds irrespective of the basic peptide structure.

The preferred use of the process described to determine ligands according to the invention is for the active centers of enzymes. The process can, however, also be transferred to other macromolecules (proteins, DNA, RNA), provided that they have suitable surfaces. The following spheres of application could be considered:

Binding molecules and/or detection molecules in diagnostic assays

Foodstuffs industry: search for ligands for flavor receptors and use as a flavor additive

Biotechnology: molecules for affinity purification

Proteins to be bound for therapeutic purposes;

enzymes, receptors, DNA, RNA

cytokines or growth factors and their receptors, particularly those involved in regulating metabolism and the immune system

cell adhesion proteins and their receptors

proteins of signal transduction pathways and their binding partners

cytosolic receptors, steroid receptors

blood-clotting proteins

neurotransmitters and their receptors

proteins of metabolic pathways

proteins involved in replication, transcription and translation

proteins of pathogens (bacteria, viruses, eukaryotic unicellular organisms, parasites), structural proteins

The process according to the invention can also be used to determine protein structures. It does not depend solely on sequence similarity but instead uses structural similarities in the molecular interfaces of secondary structural elements to predict their interaction partners. This takes into account the fact that the same (similar) interfaces can emerge even with different sequences.

By way of example, the steps for determining protein structure are described below.

In the first step, the full length of a given primary structure is “wrapped” in a repetitive secondary structure. That means that β-sheets or α-helices are calculated at standard Φ, φ and χ angles along the whole length of the primary structure.

In a second step, the molecular surfaces of the secondary structural elements that have been created are clustered and assessed with an artificial neural network, with input data derived from the molecular surfaces of the clustered structural elements. This assessment seeks on the one hand to confirm whether molecular surfaces that are representative of the given structural element can be formed in the secondary structural element with the given primary structure. If this proves not to be the case the secondary structure is rejected. This offers a new process for predicting secondary structures. The neural network is trained using known protein structures.

As an alternative to general structure formation based on standard Φ, φ and χ angles for helices or sheets, known prediction algorithms for secondary structures can be employed, with the process described above only being used for the predicted structures (parts of the sequence). The clusters found that are in contact with a particular secondary structural element (or solvent) are used in a further step to search the DIP database for the same or similar molecular surfaces and their neighbors. This is done with the bias-free superposition algorithm for atomic sets described above.

The step just described produces a series of molecular surface patches, for which a partner element is more or less definitely known (variant planning). If “non-solvent” is predicted here, a simple docking algorithm is employed in a third step to attempt to localize a suitable surface in secondary structural elements other than the one being directly considered. The simple docking algorithm is based on the fact that it is possible to search for molecular interface pairs within a particular distance from both the centers, or within a particular angle of the direction indicated. Molecular density determination is used to examine the quality of the fit (see above, Goede et al.). Once the potential partners have been determined, a fourth step involves examining the theoretical foldability whilst maintaining all the predicted neighboring components (solvent, helix-helix, helix-coil, helix-extended) and the general folding or several versions of the given sequence are adopted.

The following example seeks to elucidate the process described in the invention.

### EXAMPLE

Inhibitor Design for Proteasome

The secondary structural elements that constitute the binding site are determined, taking as a starting point the binding site for an active sub-unit of the proteasome in yeast. It transpires that five elements are involved, with two larger elements determining the binding site. Subsequently the external surfaces of these secondary structures are determined (molecular surface elements). A search is done in the DIP database for basis patches using the molecular surface elements that make up the contact and comprise 12 to 22 atoms. Similar basis patches of a particular minimum value, whereby at least 70% of the atoms are superposed and the rms value is 1.0A, are superposed with the initial surfaces, whereby the amino acids that form the counterpart, the contact patchare included in the coordinate transformation. After coordinate transformation, the basis patches found lie on the atoms of the binding sites, with the counterparts (contact patches) in the binding pocket.

The contact patches that have been found, which constitute the potential ligands, are examined to determine whether they fill the binding pocket and whether the distances from the atoms of the binding pocket are sufficiently large. The local density in the binding pocket is calculated to that end. The best potential ligands constitute the lead compounds.

Comparing the ten best potential ligands with a proteasome structure of Archaebacteria, which is available with a ligand, shows that the main chain of a structure calculated using this method is fully identical with the known inhibitor of the proteasome of Archaebacteria.

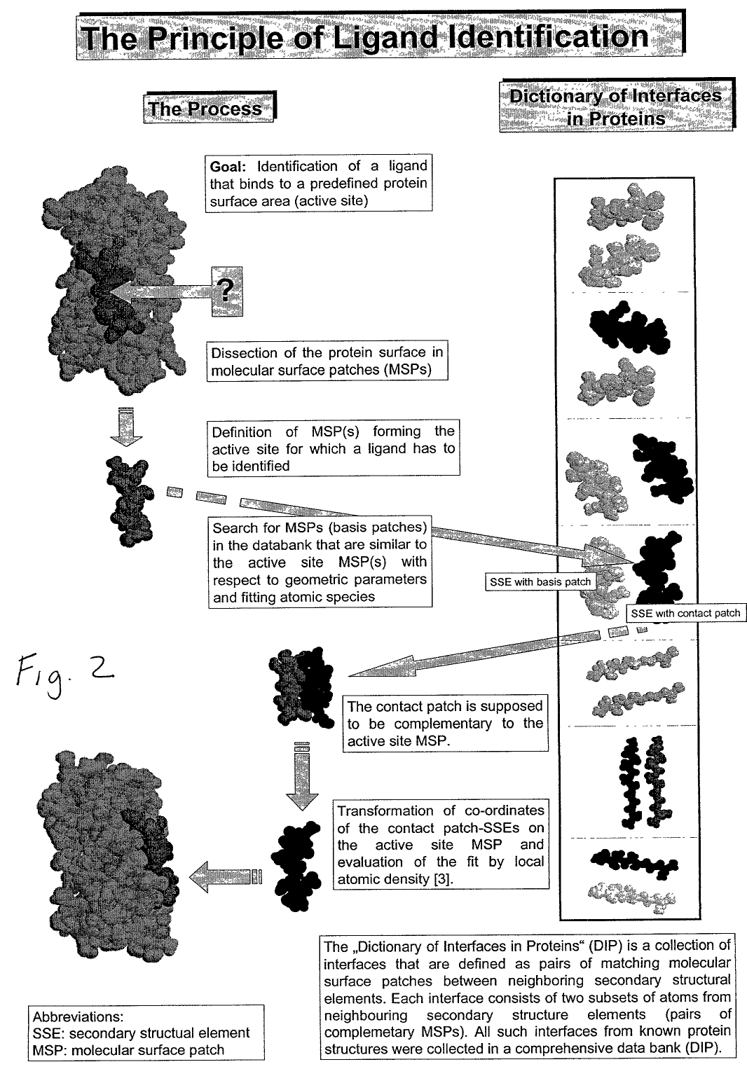

FIG. 2 further illustrates the process of ligand identification using the method of the present invention. In one aspect, the method may be used to identify ligands that bind to a predefined area of a protein molecule, DNA strand, RNA strand, or other macromolecule. The predefined area may further comprise an active site on the macromolecule wherein upon the ligand binding to the active site desirable effects are achieved. As previously discussed, the ligand binding may result in catalytic conversion of an enzyme, activation or inactivation of an enzyme, inhibition of a protein-protein interaction, conformational changes of the macromolecules or other changes which affect the physical or chemical properties of the macromolecule.

The process begins with the determination of secondary structure elements of the protein that constitute the ligand binding site. This determination is made by the dissection or decomposition of the protein surface into molecular surface patches or elements (MSPs) where the surface area of the target protein to which the ligand that has to be determined to bind is modeled as secondary structural elements derived from the target protein. This modeling process further defines the active site of the protein, for which ligands are desirably directed to bind, by one or more basis patches. The basis patches comprise surface areas of secondary structural elements made of groups of atoms that are similar to the molecular surface patches.

Following the decomposition of the protein surface, a search of the basis patches directed towards the MSP is made. A databank or database of molecular surface information information, such as the Dictionary of Interfaces in Proteins (DIP), which is composed of pairs of matching molecular patches between neighboring secondary structural element surfaces, may be used to search for suitable basis patches. Suitable database matches will have similar geometric and/or atomic fitting parameters as compared to those of the basis patches.

Subsequently, contact patches having surface areas of secondary structural elements made of groups of atoms that are in contact with the basis patch are identified. In one aspect, the contact patches are candidate selections if they are complementary to the active site MSP.

Upon identification of suitable contact patches, the co-ordinates of the contact-patch secondary structural elements are identified relative to the active site of the MSP. In one aspect, the coordinate transformation of the contact patch with respect to the molecular surface patches and the respective complementary neighboring elements is indicative of the ligand binding site with and rms value less than 2 angstroms. The results of this transformation are further evaluated by their fit, comparing local atomic and packing densities wherein a complementary neighboring element represents a compound being a potential ligand and a better fit indicates a better potential for the compound to be a ligand for the protein of interest.

What is claimed is:

1. A process for identifying compounds as potential ligands for a protein having a ligand-binding site, comprising:
a) determining secondary structural elements of the protein that constitute the ligand-binding site;
b) breaking down the molecular surface of the ligand-biding site of the protein into molecular surface elements;
(c) identifying known molecular surface patches that are complementary to a neighboring molecular surface element;
(d) effecting coordinate transformation of the molecular surface patches identified in step c) with a neighboring molecular surface element, based on a starting element at an rms value less than 2 Å;
(e) identifying counterparts of the molecular surface patches in known compounds; and
(f) assessing the fit of the compounds identified in step (e) in terms of local packing density, wherein a better fit indicates a better potential for the compounds to be ligands of the protein.

2. The process as described in claim 1, wherein external surfaces of the secondary structures of the ligand binding site are determined in step (b).

3. The process as described in claim 1 wherein the known molecular surface patches are superposed with the secondary structural elements.

4. The process as described in claim 1 wherein the molecular surface patches lie on atoms of the binding site after a coordinate transformation.

5. The process as described in claim 1, wherein the identified ligands are compared with a known initial protein plus ligand.

6. The process as described in claim 1, wherein the ligands are peptides.

7. The process as described in claim 1 wherein the proteins are enzymes.

8. The process as described in claim 1, wherein the rms value is 1.5 Å.

9. The process of claim 1 wherein the known molecular surface patches are identified from a database.

10. The process of claim 1 wherein the known neighboring element is a receptor or an enzyme.

11. The process as described in claim 6, wherein the peptides comprise at least 10 amino acids.

12. The process as described in claim 6 wherein the peptides are subsequently transformed into a peptidomimetic.

13. A process of determining the structure of a protein, comprising: identifying ligands from known molecular surface patches using the process of claim 1; and determining the structure of the protein based on the known structure of the neighboring element to which the molecular surface patches bind.

14. The process as described in claim 2, wherein the molecular surface patches create contact with said external surfaces.
